(19) United States
(12) Patent Application Publication
(10) Pub. No.: US 2017/0071580 A1
(43) Pub. Date: Mar. 16, 2017

(54) System and method for non-invasive monitoring

(71) Applicant: Alex Artsyukhovich, Irvine, CA (US)

(72) Inventors: Alex Artsyukhovich, San Juan Capistrano, CA (US); Lingfeng Yu, Rancho Santa Margarita, CA (US)

(73) Assignees: Alex Artsyukhovich, Irvine, CA (US); Lingfeng Yu, Rancho Santa Margarita, CA (US)

(21) Appl. No.: 14/998,690

(22) Filed: Feb. 2, 2016

Related U.S. Application Data
(60) Provisional application No. 62/125,997, filed on Feb. 3, 2015.

(51) Int. Cl. A61B 10/00; A61B 5/00; A61B 5/0205; A61B 5/145

(52) U.S. Cl. CPC A61B 10/00; A61B 5/14507; A61B 10/0051; A61B 5/7282; A61B 5/02055; A61B 2010/0087; A61B 5/082

(57) ABSTRACT

This invention describes a method and device for big data approach in integrated patient care. It combines breath and saliva chemical analysis with physical signs analysis to use big data analysis for diagnosing a disease state based on the determined concentration of the group of gas compounds and physical parameters.

### BACKGROUND

Sepsis is a global healthcare problem. It is more common than heart attack, and claims more lives than any cancer. There were 1,141,000 US sepsis cases in 2008 and the number of sepsis cases grows each year by ˜12%. There were 207,427 deaths from sepsis in US in 2007. In the least developed countries, sepsis remains a leading cause of death. Many doctors see sepsis as 3 stage phenomenon, starting with sepsis and progressing through severe sepsis to septic shock, often resulting in death of a patient.

Currently sepsis is detected by Doctor looking at physical signs of the patient. The only analysis confirming sepsis is drawing blood and waiting for bacterial culture to grow—this takes hours. Blood drawing is invasive, it weakens sepsis patients, who are typically in need of larger blood volume and are being on IV with fluid pumped in patient's cardio-vascular system. There are no non-invasive live sepsis-monitoring medical devices available for purchase in USat this moment.

### SUMMARY

In this invention, we propose a path to producing non-invasive live monitoring medical device for sepsis. We plan to analyze gas content in patient's inhaled and exhaled breath and detect content changes strongly indicative of sepsis. We plan to also complement this method with other physical signs sensors to strengthen sepsis diagnosis:
1. Pulse rate meter
2. Breathing rate meter
3. Thermometer
4. Blood pressure meter
5. Tremor meter
6. humidity meter
7. Oxygen saturation (?)

Breath can be directed by patient in breathalyzer type of device. Or it can be continually monitored at exhaust end of the ventilator or other artificial breathing apparatus.

The use of the sepsis monitor can be in various scenarios:
1. Hospital ER/Triage Rooms for screening newly admitted patients
2. Hospital rooms for monitoring sepsis condition in hospital patients who are often on ventilators
3. Doctor's offices for monitoring inflammation and improvements with different treatment plans
4. Home use for patients with chronic inflammatory conditions to warn of early inflammation flares, that can turn into sepsis

### BRIEF DESCRIPTION OF THE DRAWINGS

FIG. 1 depicts schematically one proposed implementation of suggested device and method as a block scheme. It shows breath analyzer, saliva collector and analyzer and physical signs sensors—all data collected in processing unit for analysis.

FIG. 2. depicts one possible device hand held implementation for home, office and ER screening.

FIG. 3 depicts another implementation of the proposed device for hospital rooms and patients on ventilators (artificial lung machines).

FIG. 4. depicts one possible implementation of the device and method utilizing Mid IR Broadband Source spectroscopy for breath analysis.

FIG. 5 depicts hand held monitoring/diagnostic device based on Mid IR laser spectroscopy.

### DETAILED DESCRIPTION OF THE DRAWINGS

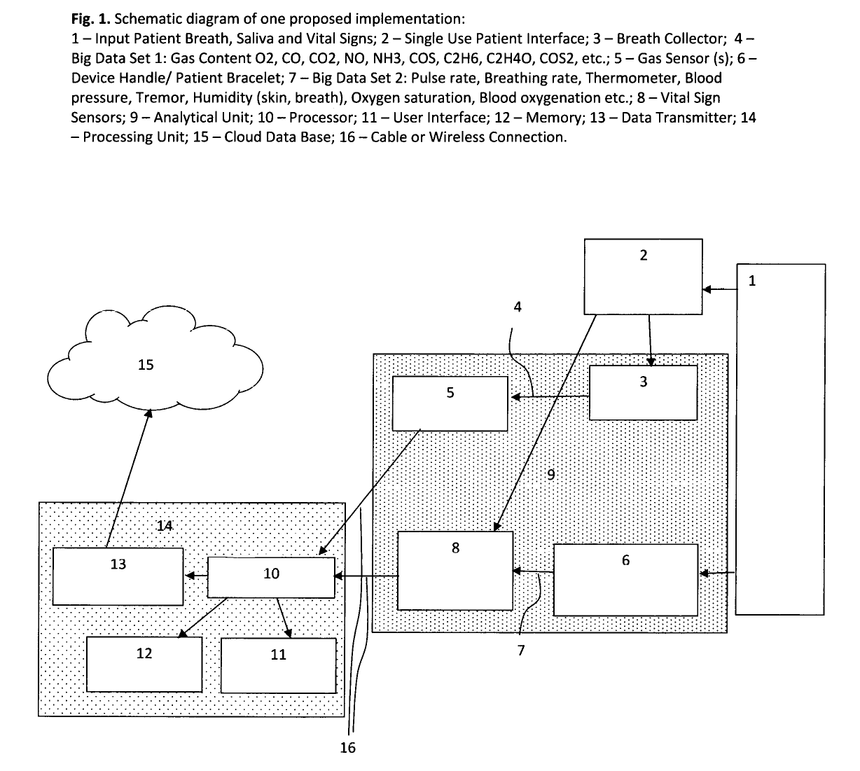

FIG. 1 depicts schematically one proposed implementation of suggested device and method. One skilled in this art can propose other implementations that are essentially equivalent in function but differ in diagram, but do not alter the intent of invention.

Breath of the patient (1) is channeled throw single use interface (2) into breath collector (3). This can be done either by patient exhaling into single use interface (2) or by breath collector (3) having a small pump or vacuum line integrated for active exhaled air (1) collection. The exhaled air contains various gases and vapors—organic and inorganic (4): O2, CO, CO2, H2O, NO, NH3, COS, C2H6, C2H40, COS2, C2H5OH, etc. These gases and vapors are analyzed in content and concentration via gas sensor(s) (5) generating big data set 1 (4). The gas and vapor sensors can be different in nature—UV/VISIBLE/IR spectroscopy, laser spectroscopy, gas chromatography, thermal conductivity, electro-chemical, chemical, etc. The type and nature of gas sensor does not affect the intent of this invention and simply allows for particular implementation of the invention. The vital signs of patient (7) are the following: pulse rate, breathing rate, temperature, blood pressure, tremor, humidity (skin, breath), blood oxygenation, etc. These vital signs (7) are collected through additional sensors in Single Use Patient Interface 2, Breath Collector 3 and/or device handle/patient bracelet (6). They are analyzed by vital signs sensors (8) and create a second big data set (7). Both big data sets 4 and 7 are analyzed by device processor (10). The results are then displayed for user on graphic user interface—GUI (11). This data set, comprising a snapshot in time of patient's condition is then stored in memory of the device (12) and transmitted by a transmitter (13) into a cloud (15). The processing unit (14), comprising of processor (10), GUI (11), memory (12), transmitter (13) can be a tablet computer, laptop, smart phone or a custom device. The analytical unit (9), comprising of breath collector (3), gas sensor(s) (5), device handle/patient bracelet (6) and vital sign sensors (8), can be integrated with processing unit (14) or be a separate unit, connected to processing unit (14) with cable or wirelessly. The single use interface (2) is a throw away item for hygienic use of the device and equipped with saliva collector. It may contain sensors for vital signs (like thermocouple) or for saliva chemical analysis in its saliva collector.

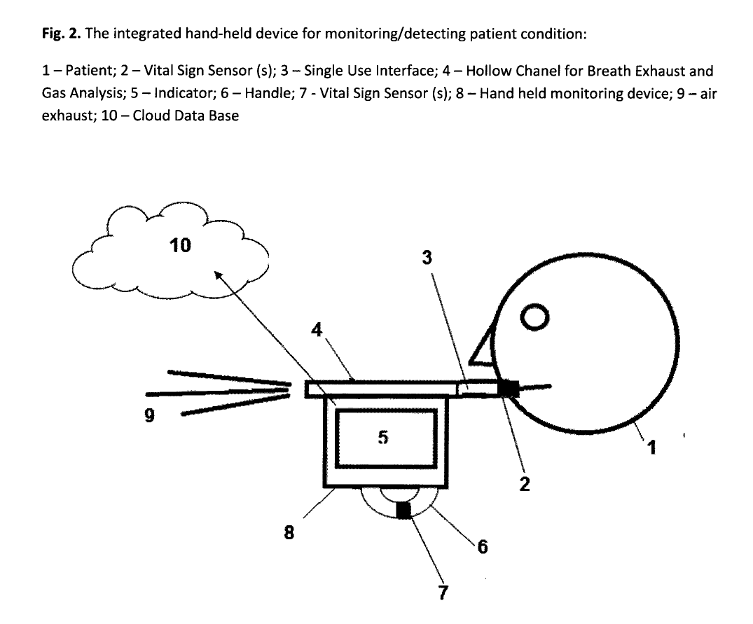

One possible device implementation for home, office and ER screening is shown in FIG. 2. In it patient blows through single use interface attached to a hollow tube. The analytical unit with gas sensor and vital sign sensors is integrated with compact processing unit, for example a smartphone. Vital signs are measured through patient contact of lips by single use interface (Vital Signs Sensor(s) 1) and patient's hand by device handle sensors (Vital Signs Sensor(s) 2). The single use saliva collector can be equipped for chemical and other sensors for saliva analysis. In one implementation the gas sensor can be an infrared spectrometer for gas IR absorption analysis. In another implementation gas analysis can be done by electrochemical sensors. The nature of gas sensor is immaterial to a subject of this invention. The integrated device display can display values of gas concentrations and vital signs, or their integrated derivatives, characterizing general state of patient. These derivatives can be established for example through multiple parameter regression of patient's blood analysis with big data sets, collected by a proposed device.

As an example, the methods that can be used for gas analysis of breath may include, but not limited to:
1. Gas chromatography
2. Absorption spectroscopy (UV, Visible or IR)
3. Fluorescence
4. Thermal conductivity
5. Electro-chemical sensors

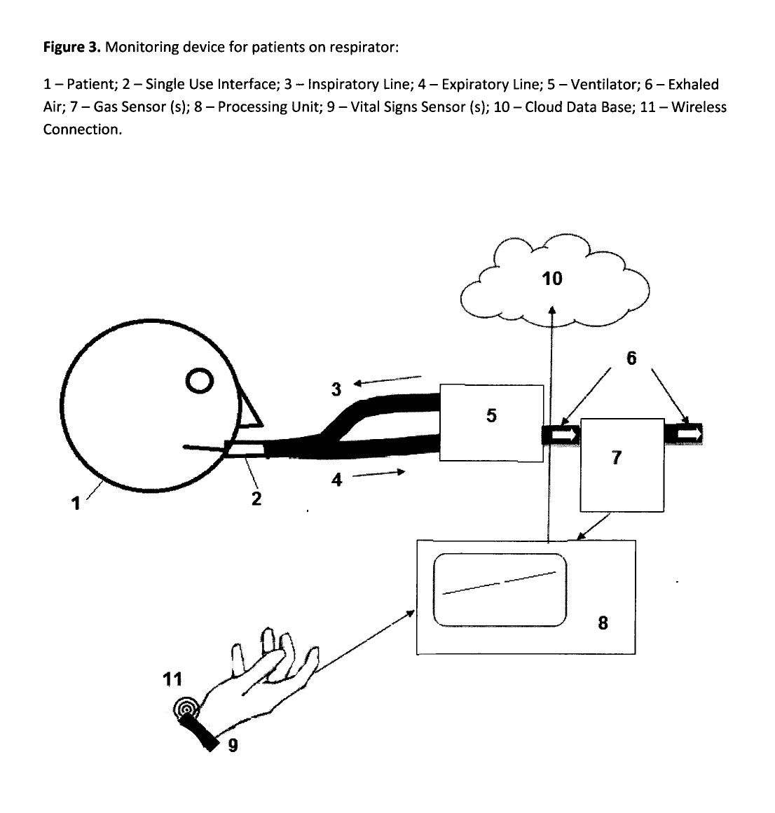

FIG. 3 shows another implementation of the proposed device for hospital rooms and patients on ventilators (artificial lung machines). In this case the device is mounted to a ventilator exhaust as monitor of patient condition. The vital signs sensors are integrated in patient's bracelet that communicates through a cable or wirelessly with processing unit. The processing unit analyzes data, displays it on GUI and sends it to cloud.

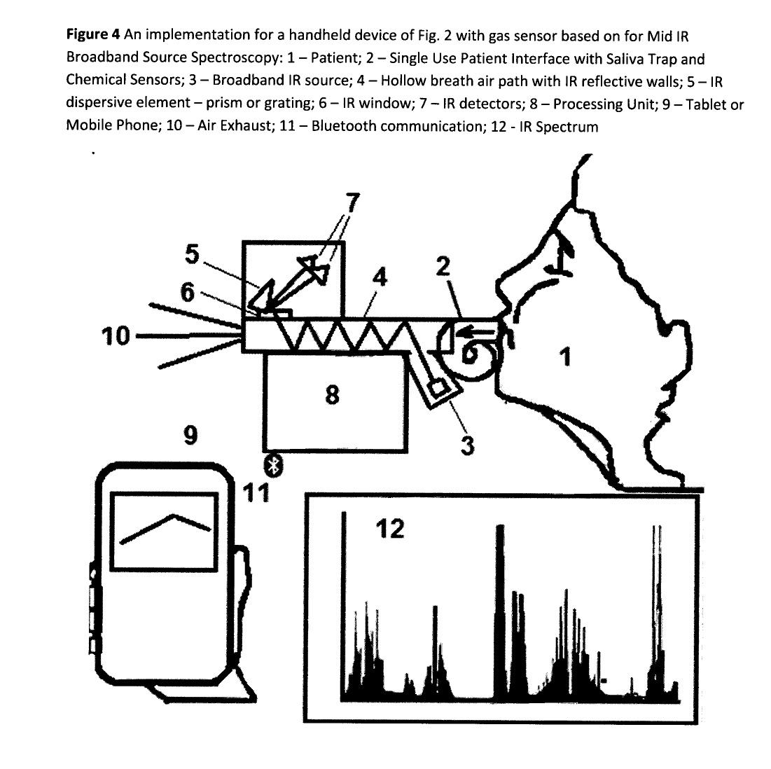

FIG. 4. Mid IR Broadband Source spectroscopy based handheld monitoring and diagnostic device. The IR spectrum in the bottom right corner exhibit air spectrum with certain gases absorption lines, for example CO and CO2. A modulated broadband source injects mid IR (1-5 um) into mid IR reflective light-pipe for multi-pass Mid IR absorption spectroscopy. The light pipe can be also replaced by an integrating sphere, made with Mid IR reflective material, like copper or gold. After multiple reflections in the light pipe the light is directed towards spectral analysis element—spectrally selective filters, dispersive prisms or diffractive gratings. It is then directed to a line of Mid IR sensors, positioned to measure mid IR intensity at absorption peaks of different gases. The mid IR radiation passes multiple time through air exhaled by patients and thus mid IR radiation is absorbed at wavelengths, characteristic of specific gases, like CO, CO2, H2O, etc. The signal of the gas sensors (mid IR sensors) is than processed and displayed on hand held unit or transmitted to processing unit (smartphone in this implementation) for filing and displaying the historic monitoring charts. The vital signs sensors can be located in single use patient interface, device handle or smartphone (accelerometer for tremor measurement). The modulation of IR source intensity allows for higher sensitivity, due to background light rejection.

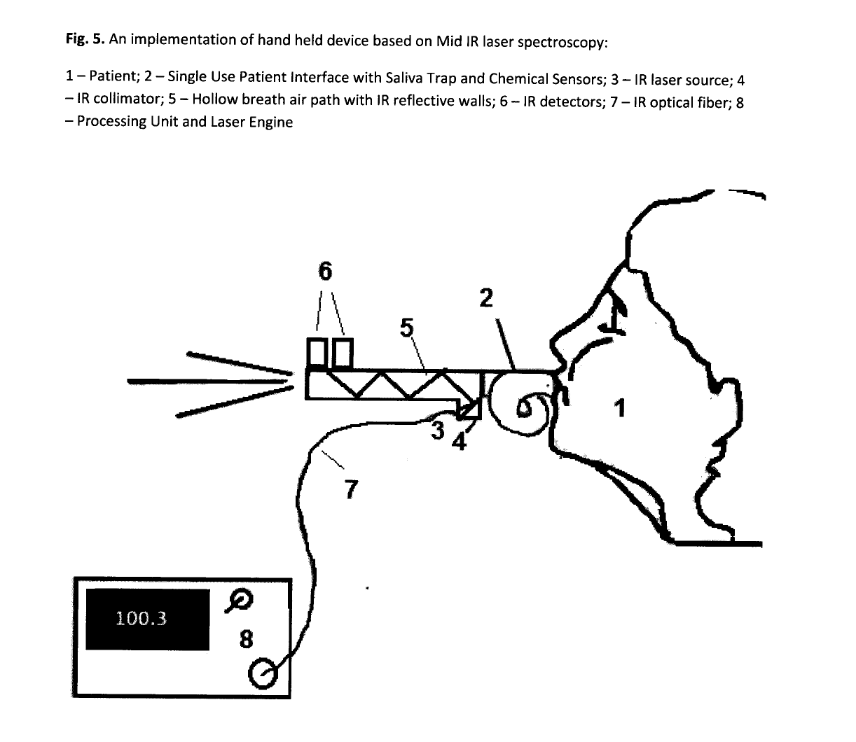

FIG. 5 is showing hand held monitoring/diagnostic device based on Mid IR laser spectroscopy. The design is similar to one shown above in FIG. 4, but it uses mid IR lasers for mid IR absorption spectroscopy. In this case just a couple of lasers are used—each has laser wavelength centered on particular gas mid IR absorption peak, for example CO and CO2. The two mid IR sensors may have mid IR spectrally selective filters installed in front of them to separate two wavelengths. Any other spectrally dispersive elements can be used for this purpose—dispersion prisms, diffraction gratings and such.

1. A method comprising:
collecting a sample of exhaled breath from a subject;
collecting a sample of saliva for chemical analysis;
collecting physical parameters from a subject;
determining concentrations of a group of chemical compounds in the collected sample; and
diagnosing a disease state based on the determined concentration of the group of chemical compounds and physical parameters.

2. A method comprising:
collecting a set of samples of exhaled breath and physical parameters from a subject over a
period of time; determining normalized concentrations of a group of chemical compounds in
each collected sample of exhaled breath and saliva;
determining a change in the normalized concentrations of the group of chemical compounds over the period of time; determining a change of physical parameters over the period of time; and
evaluating an disease state of the subject based on the determined change in the normalized concentration of the group of chemical compounds and physical parameters.

3. A device, comprising:
a breath collector configured to receive exhaled breath from a subject;
one or more sensors configured to output a concentration of a group of gas compounds in the inhaled breath and output a concentration of said group of compounds in the received exhaled breath; and
one or more sensors configures to detect physical parameters (including at least one of the Pulse Rate, Breathing rate, Thermometer, Blood pressure, tremor and humidity parameters) from the subject; and
one or more sensors configures to detect chemicals in collected samples of saliva, and
a processor operably coupled to the one or more sensors and further configured to take parameters of said gas compounds, saliva chemical composition and physical parameters as input and calculate one or more indicative parameters, using big data approach

4. A device of previous claim,
using a single-use patient interface configured to collecting exhaled breath and saliva from a subject
